(19) United States
(12) Patent Application Publication
(10) Pub. No.: US 2025/0066831 A1
(43) Pub. Date: Feb. 27, 2025

(54) TEMPERATURE-RESPONSIVE NANO-BIOMATERIALS FROM GENETICALLY ENCODED FARNESYLATED PROTEINS

(71) Applicants: Shahadat Hossain, Syracuse, NY (US); James Hougland, Syracuse, NY (US); Davoud Mozhdehi, Syracuse, NY (US)

(72) Inventors: Davoud Mozhdehi, Syracuse, NY (US); Shahadat Hossain, Syracuse, NY (US); James Hougland, Fayetteville, NY (US)

(73) Assignee: Syracuse University, Syracuse, NY (US)

(21) Appl. No.: 18/722,636

(22) PCT Filed: Dec. 21, 2022
(86) PCT No.: PCT/US22/82119

§ 371 Date: Jun. 21, 2024

Related U.S. Application Data
(60) Provisional application No. 63/292,234, filed on Dec. 21, 2021.

(51) Int. Cl. C12P 21/02; C12N 9/10; C12N 15/70; C12P 7/64

(52) U.S. Cl. CPC C12P 21/02; C12N 9/1085; C12N 15/70; C12P 7/64; C12Y 205/01058; C12Y 205/01059; C12N 2800/101

(57) ABSTRACT

A prokaryote was genetically engineered to develop operationally simple, high-yield biosynthetic route for the production of farnesylated proteins. The recombinant organism was modified to express a target protein, a peptide sequence fused to the target protein at a C-terminus, and an alpha and a beta subunit of a prenyltrasferase. The prenyltrasferase may be farnesyltransferase or geranylgeranyl transferase, and the peptide sequence may comprise cysteine, two hydrophobic amino acids, and an amino acid having selectivity to farnesyltransferase or geranylgeranyl transferase.

### CROSS-REFERENCE TO RELATED APPLICATIONS

The present application claims priority to U.S. Provisional Application No. 63/292,234, filed on Dec. 21, 2021.

### STATEMENT REGARDING FEDERALLY SPONSORED RESEARCH OR DEVELOPMENT

This invention was made with government support under Grant Nos. R01GM132606 and R35GM142899 awarded by the National Institutes of Health (NIH) and Grant No. 2105193 awarded by the National Science Foundation, Division of Materials Research. The government has certain rights in the invention.

### INCORPORATION-BY-REFERENCE OF MATERIAL SUBMITTED ON A READ-ONLY OPTICAL DISC, AS A TEXT FILE OR AN XML FILE VIA THE PATENT ELECTRONIC SYSTEM

The contents of the electronic sequence listing (156P663sequence.xml; Size: 12,521 bytes; and Date of Creation: Jun. 20, 2024) is herein incorporated by reference in its entirety.

### BACKGROUND OF THE INVENTION

### 1. Field of the Invention

The present invention related to lipidated protein complexes and, more specifically, to an approach for producing farnesylated proteins from genetically engineered bacteria.

### 2. Description of the Related Art

The development of new protein-based biopharmaceuticals and materials is a vibrant area of research at the interface of chemistry, biology, and materials science and engineering. Efforts in this space have traditionally focused on engineering the amino acid sequence and structure of proteins to achieve a specific function for applications including biomaterial, sensors, and biocatalysts, among others. Instead of changing the sequence of proteins, cells leverage an alternative strategy, post-translational modification (PTMs), to modulate a protein's structure and function with exquisite spatiotemporal control. Despite the significant interest in the biology of PTMs, this strategy remains underutilized for diversifying physicochemical properties, engineering capabilities, and/or the biological behavior of proteins. This lacuna exists because the synthesis of proteins with compositionally defined PTM patterns remains challenging, and although more than 300 PTMs have been identified, the energetic interplay and (bio) material consequences of only a handful of these modifications have been systematically studied.

Lipidation is a common PTM in eukaryotes where the nature of the attached lipid (e.g., fatty acids, sterols, isoprenoids, etc.) dictates biological outcomes by controlling protein structure, function, and localization. For instance, isoprenylation of Ras signaling proteins—the modification of cysteine residues with either 15 carbon (farnesyl) or a 20 carbon (geranylgeranyl) isoprenoid lipid—is critical for their localization to correct membrane-bound organelles, as well as regulating their function. While the biology of isoprenylation and the interactions between prenylated proteins and lipid bilayer are of broad research interest, application of this PTM to control the assembly and function of protein-based materials remains underexplored. This is due in large part to the lack of a facile, scalable, and inexpensive synthetic method to lipidate proteins. Many lipidated proteins (LP) cannot be produced by genetic code expansion methods due to the stringent preference of ribosomes for amino acid-derived motifs. Alternatively, their multi-step convergent semi-synthesis is laborious and technically challenging. The inability to rapidly generate LPs has hindered the empirical elucidation of sequence-structure-function rules and computational parametrization of LPs' design space. Accordingly, there is a need in the art for an operationally simple, high-yield biosynthetic route to produce prenylated proteins.

### BRIEF SUMMARY OF THE INVENTION

The present invention is an operationally simple, high-yield biosynthetic route to produce prenylated proteins by genetically engineering E. coli to co-express the desired protein and the minimum enzymatic machinery required for prenylation. In eukaryotes, isoprenylation is carried out by specialized transferases that bind to a lipid donor (isoprenyl pyrophosphate) and modify a recognized peptide substrate fused to the C-terminus of target proteins. The present invention focuses on farnesylation because E. coli can biosynthesize farnesyl pyrophosphate (FPP) from the sequential condensation reactions of dimethylallyl pyrophosphate with two molecules of 3-isopentenyl pyrophosphate. This endogenous metabolic pathway provides access to farnesyl lipid donors without the need for genetic/metabolic engineering. The next requirement for the construction of the minimal recombinant expression system for protein farnesylation is the identification of appropriate prenyl transferase enzyme(s) that can be heterologously expressed in an active form in bacteria. Three major classes of prenyl transferases are known: farnesyltransferase (FTase) and geranylgeranyl transferase (GGTase) types I and II. The present invention included an investigation into the potential utility of FTase and GGTase-I due to their broad substrate scopes: proteins fused to the “CaaX” (SEQ ID NO: 1) motif at the C-terminus. In this motif, the cysteine, which is the site of PTM, is followed by two hydrophobic amino acids; and the final amino acid (X) determines preferential selectivity toward FTase (X=Ser) or GGTase-I (X=Leu).

To reveal how farnesylation modulates the assembly and properties of proteins, elastin-like polypeptides (ELPs) were as a model system. ELPs are artificial peptide polymers that are derived from the consensus sequence of tropoelastin (GZGVP), in which the guest reside (Z) can be any amino acids other than proline. ELP was chosen as model system for two reasons: 1) ELPs have a well-characterized lower critical solubility transition (LCST) behavior in which they undergo a soluble-to-insoluble temperature above a critical transition temperature (Tₜ). In addition to solution parameters (e.g., concentration, ionic strength, pH, etc.), this Tₜ depends on the molecular syntax of the ELP (e.g., the hydrophobicity of the guest residue, the length of the polypeptide) and the physicochemistry of PTM motif. The phase-separation behavior of ELP can be conveniently monitored using various scattering techniques and therefore enable us to parse the effect of farnesylation on the thermo-response of lipid-protein conjugates. 2) ELPs can be expressed at high yields in E. coli and can be purified at scale using non-chromatography techniques that leverage their reversible temperature-triggered phase behavior. Additionally, the stimuli-responsive characteristics of ELPs enable the fine-tuning of the emergent assembly of farnesylated protein with temperature.

An embodiment of the present invention thus includes a recombinant organism for forming a lipidated protein, where the recombinant organism is a host organism that has been modified to include a first gene expressing an alpha subunit and a beta subunit of a transferase that will attach a lipid from a lipid donor to a protein. The host organism is further modified to include a second gene that expresses the protein, wherein the protein includes a substrate of the transferase. The transferase may be selected from the group consisting of farnesyltransferase and geranylgeranyl transferase. The substrate of the transferase may comprise a first amino acid that is cysteine, a second amino acid that is hydrophobic, a third amino acid that is hydrophobic, and a fourth amino acid that has selectivity to farnesyltransferase or geranylgeranyl transferase. The host may be capable of endogenously producing the lipid donor. The lipid donor may comprise farnesyl pyrophosphate. The substrate of the transferase may be selected from the group consisting of SEQ ID NO: 2 and SEQ ID NO: 3. The alpha subunit and the beta subunit may be translationally coupled, such as by a stop codon of the beta subunit overlapped with a start codon of the alpha subunit. The host organism may comprise a bacterium such as E. coli.

In another embodiment, the present invention may be a method of producing a lipidated protein. In one step, the method involves modifying a host organism to include a first gene expressing an alpha subunit and a beta subunit of a transferase that will attach a lipid from a lipid donor to a protein. In another step, the method involves modifying the host organism to include a second gene expressing the protein, wherein the protein includes a substrate of the transferase. In a further step, the method involves providing a lipid donor. In an additional step, the method involves culturing the host organism to express the first gene and the second gene in the present of the lipid donor to form the lipidated protein. The transferase may be selected from the group consisting of farnesyltransferase and geranylgeranyl transferase. The substrate of the transferase may comprise a first amino acid that is cysteine, a second amino acid that is hydrophobic, a third amino acid that is hydrophobic, and a fourth amino acid that has selectivity to farnesyltransferase or geranylgeranyl transferase. The host may be capable of endogenously producing the lipid donor. The lipid donor may comprise farnesyl pyrophosphate. The substrate of the transferase may be selected from the group consisting of SEQ ID NO: 2 and SEQ ID NO: 3. The alpha subunit and the beta subunit may be translationally coupled, such as by a stop codon of the beta subunit overlapped with a start codon of the alpha subunit. The host organism may comprise a bacterium such as E. coli.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a flowchart of an approach to engineer recombinant farnesylation platforms according to the present invention.

FIG. 2 is an SDS-PAGE gel analysis of FTase expressed from a pETDuet-1 vector. The M_{w} of alpha and beta subunits are 44 and 48.6 kDa. FTase was predominantly present in the inclusion bodies (i.e., the insoluble pellet of cell lysate).

FIG. 3 is a graph of In vitro farnesylation of ELPs with the soluble transferase (produced as a translationally coupled heterodimer) indicates that the recombinantly expressed enzyme is active. The reaction mixture contained 3 μM of protein substrate (ELP-CVLS), 0.2 μM FTase, and 10 μM of farnesylpyrophosphate (FPP), and 5 mM TCEP in HEPPSO buffer (50 mM, pH=7.8). The reaction mixture was incubated at room temperature for 16 h before analysis by RP-HPLC on a C18 column using a linear gradient of acetonitrile in water (0-90% over 40 min). The negative control lacked the FPP lipid donor.

FIG. 4 is a series of schematics and graphs showing: (a) The architecture of plasmids used for the biosynthesis of farnesyl-modified ELPs. Two compatible plasmids were used to encode all genetic elements necessary for in vivo farnesylation: ELPs fused to two canonical CaaX motifs, CVLS and CVLL; a and b subunits for FTase or GGTase-I which together constitute the heterodimeric prenyl transferase. These subunits were expressed independently (from the bicistronic pETDuet-1 vector) or translationally coupled by including ribosomal binding site and start codon (underlined) for the α-subunit at the end of the b-subunit. (b) Representative RP-HPLC chromatogram for (V/A)₈₀ (fused to CVLL) expressed in the absence (dashed black line) or the presence (solid red line) of prenyl transferase. The peak marked with an arrow corresponds to the dimer of (V/A)₈₀, formed via a disulfide bond between the cysteine residue in CaaX motif (SEQ ID NO: 1). (c) Comparison between the cumulative yields of farnesylated-protein produced from co-expression of (V/A)₈₀ fused to CVLX (X=Ser or Leu) and FTase or GGTase-I. While FTase can farnesylate (V/A)₈₀ fused to either substrate, GGTase-I can produce the highest yield of (V/A)₈₀-Fr from substrate bearing CVLL peptide, 2-3×the amount produced by the canonical enzyme (FTase). Error bars in c represent standard deviations of 2-5 independent replicates. One-way ANOVA followed by Turkey's HSD test, *, ***, and **** signify p<0.05, 0.001, and 0.0001.

FIG. 5 is a series of graphs of (a) Reverse-phase HPLC chromatograms of unmodified and farnesylated V₄₀ and (V/A)₈₀ confirms the purity of each construct, and increased hydrophobicity of farnesylated proteins. (b,c) The MALDI-TOF-MS analysis is consistent with the addition of a single farnesyl group to each protein. (d) The location of farnesyl group is confirmed by the digestion of the (V/A)₈₀-Fr with trypsin and the analysis of peptide fragments using MALDI-TOF-MS. The molecular weight of the C-terminus peptide fragment (GCVLL) is increased by 205.2 Da, corresponding to the mass of Fr group. (e) 1H NMR spectra of V₄₀ and V₄₀-Fr in D₂O and DMSO-d6. The broadening of V₄₀-Fr peaks in D₂O is due to the lipid-induced oligomerization of V₄₀-Fr. The green band highlights the position of allylic methyl groups in farnesyl group. (f) Attenuated total reflectance FT-IR spectra of the amide (I-III) region of V₄₀ and V₄₀-Fr. The broad bands in this region are consistent with the presence of large fraction of disordered domains in the lyophilized state of both proteins, showing that farnesylation does not alter the structure of ELP at the chain-level.

FIG. 6 is the assigned 1H NMR spectra of V₄₀-Fr in DMSO-d6 (2.5 ppm). The peak at 3.3 ppm is residual water.

FIG. 7 is a series of graphs of the characterization of the effect of farnesylation on ELP's thermo-response in PBS using turbidimetry and differential scanning calorimetry. (a) Turbidity profiles of unmodified (dashed line) and farnesylated (solid line) V₄₀ and (V/A)₈₀, black and blue lines respectively. Dotted line represents the standard deviations of two independent measurements. (b) Temperature-programmed turbidimetry is used to monitor the reversibility of the LCST phase-transition of V₄₀-Fr (solid line) and (V/A)₈₀-Fr (dashed line) after one cycle of heating and cooling. Both lipidated proteins showed complete reversibility in turbidity after heating above Tₜ and cooling down below Tₜ. Farnesylation does not alter the reversibility of ELP phase separation. [protein]=6 μM in a and b. (c) The concentration dependence of the proteins' transition temperatures. The data for Tₜ vs. natural log of protein concentration is fitted using a linear regression model. Dotted lines represent a 95% confidence interval for the fitted line. (d) The DSC thermograms of the unmodified (dashed curves) and farnesylated (solid line) proteins, V₄₀ (blue) and (V/A)₈₀ (black). The endothermic peak corresponds to the temperature-triggered phase separation of each construct. Farnesylation reduces the AUC but increases the asymmetry of the peak. (e) The comparison of thermodynamic parameters (enthalpy and entropy of the phase separation) for each construct obtained from the DSC analysis. Farnesylation reduces the enthalpy and entropy of phase separation of ELPs (two-tailed unpaired t-test, *p<0.05, **p<0.01). The error bars are standard deviations of two independent measurements.

FIG. 8 is a graph showing that the temperature-triggered phase transition of V₄₀ and (V/A)₈₀ is reversible.

FIG. 9 is a graph of the concentration-dependent turbidimetry analysis of unmodified and farnesylated ELPs. (a) V₄₀, (b) (V/A)₈₀, (c) V₄₀-Fr, and (d) (V/A)₈₀-Fr. Dotted lines indicate the standard deviation of two independent measurements at each concentration.

FIG. 10 is a series of graphs of the characterization of nano-assembly of farnesylated proteins in PBS using dynamic light scattering and cryo-TEM. (a) Autocorrelation functions of unmodified (dashed curve) and farnesylated (solid) constructs at 20° C. (blue—V₄₀, black—(V/A)₈₀ constructs). ACFs shows the evidence of forming micelles with farnesylated proteins while unimer is observed with unmodified proteins at 20° C. (b) Intensity based size distribution of V₄₀-Fr and (V/A)₈₀-Fr measured at 3 different scattering angles (13°, 90°, 173°) by MADLS. The vertical scale bar corresponds to 10%. (c) The hydrodynamic size of the proteins derived from the cumulant method. The unmodified proteins size corresponds to the size of unassembled unimers below Tₜ, while all lipidated samples formed larger assemblies. [Protein]=6 μM in a-c. Cryo-TEM images of V₄₀-Fr (d) and (V/A)₈₀-Fr (e) dissolved in PBS at 100 μM. V₄₀-Fr forms spherical micelles (diameter=13.3±3.0 nm) whereas (V/A)₈₀-Fr formed smaller micelles (diameter 9.1±2.7 nm), which further assembled to form supra-particles. The error bands (a) and bars (c) represent standard deviation of 6 measurements (2 independent samples, each measured in triplicate). The error bars in b are standard deviations of 3 measurements at each angle.

FIG. 11 is a graph of the size-intensity distribution of various constructs at 20° C. (blue) and 65° C. (red), below and above Tₜ respectively. All constructs form micron-size coacervates. [protein]=6 μM in PBS.

FIG. 12 is a graph showing that DLS confirms the reversible temperature-triggered phase-separation of unmodified and farnesylated proteins. The average hydrodynamic of proteins were measured at 20° C. (blue symbols and bars), and then after one cycle of heating and cooling (20° C.→65° C. (above Tₜ)→20° C.), red symbols and bars. No statistically significant differences between the average hydrodynamic radius of constructs before and after heating was observed (two-tailed nested t-test, e.g., for V₄₀-Fr t (10)=0.07, p=0.93). [protein]=6 μM in PBS.

FIG. 13 is a series of graphs of the size distribution of assemblies of V₄₀-Fr (a) and (V/A)₈₀-Fr (b) micelles obtained from the analysis of cryo-TEM images. (c) A violin plot showing the size-distribution of particles' diameter. The horizontal dashed line denotes the median value, and dotted lines denote lower and upper quarterlies. V₄₀-Fr formed larger assemblies compared to (V/A)₈₀-Fr (Two-tailed unpaired t-test, t (270)=11.7, p<0.0001).

FIG. 14 is a pair of correlograms of VA80-S/L-Fr (a) and V40-S/L-Fr (b) showing the impact of particular constructs on self-assembly.

### DETAILED DESCRIPTION OF THE INVENTION

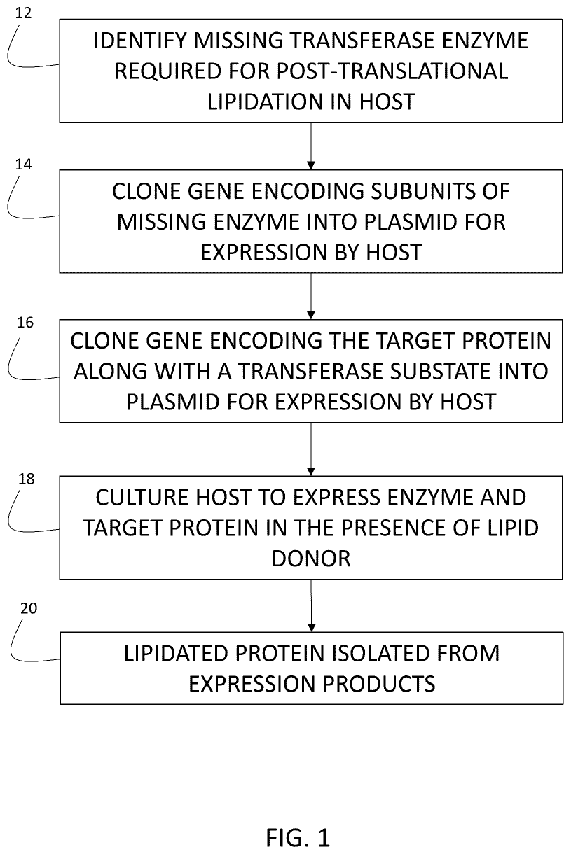

Referring to the figures, wherein like numerals refer to like parts throughout, there is seen in FIG. 1, a method 10 of engineering recombinant farnesylation platforms that can easily synthesize post translationally modified lipoproteins. Method 10 commences with an identification 12 of the missing enzymes of the host, such as E. coli, that are required for the synthesis or transfer of lipid donors to the desired protein. Next, the missing enzymes are cloned into plasmids 14 for recombinant expression in the host bacteria. A cloned gene encoding the target protein along with a substrate of the selected transferase enzyme is also cloned 16 into orthogonal plasmids for co-expression by the host. Finally, the host is cultured 18 for expression of the target protein and the transferase enzyme in the presence of a lipid donor for high-yield heterologous production of the desired lipidated products, which may then be purified from the expression results 20,

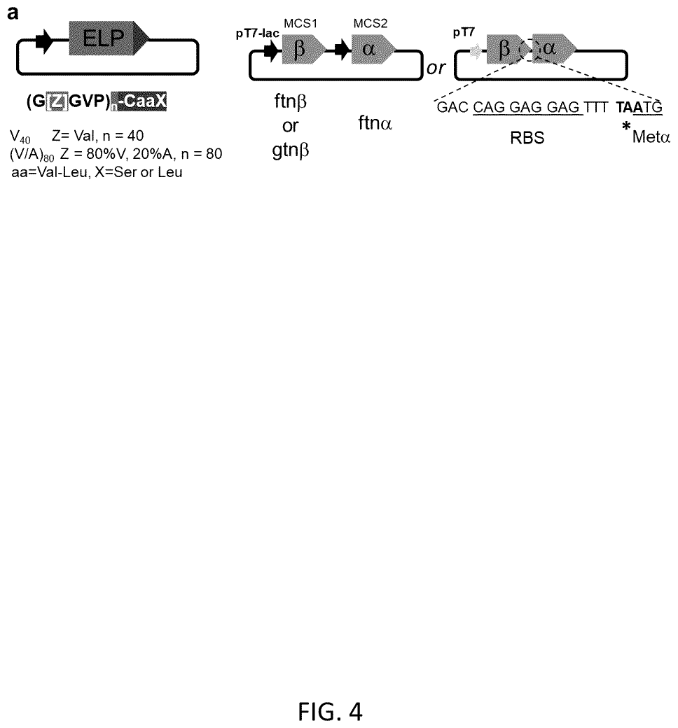

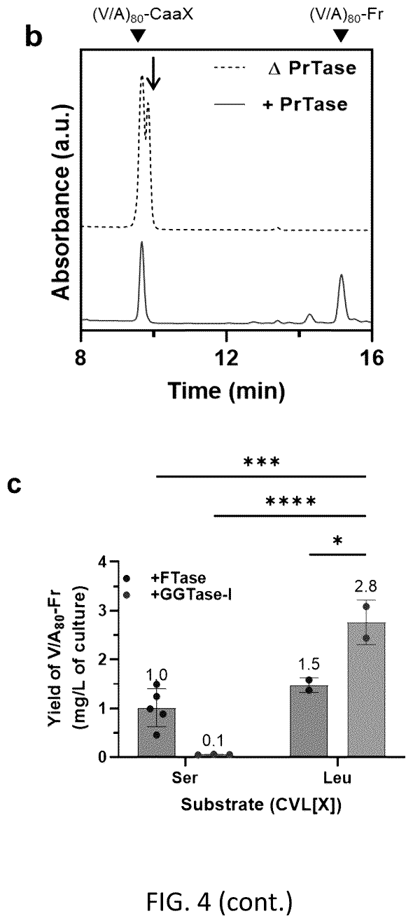

The present invention employed an iterative process to reconstitute and optimize the protein-farnesylation platforms. As a proof-of-concept example, endogenously produced FPP was used as a lipid donor to minimize the number of exogenously expressed proteins to three: a model protein (i.e., ELP) was fused to the CaaX sequence (SEQ ID NO: 1), and the alpha and beta subunits of FTase or GGTase-I. Both FTase and GGTase-I are heterodimeric proteins with an identical a subunit but divergent β subunits. FPP is the canonical lipid donor for FTase, but it is also accepted by GGTase-I. Two prenyl transferase subunits were selected from R. norvegicus as they have been previously expressed in E. coli. Genes encoding a and β subunits as well as a model ELP fused to short peptide sequences, CVLS (SEQ ID NO: 2) and CVLL (SEQ ID NO: 3) were cloned into a set of orthogonal plasmids with compatible origins of replication (pBR322 and p15A) and selection markers (Amp′ and Cm′) using Gibson assembly and recursive directional ligation and their identity were verified by DNA sequencing (FIG. 4a). Two ELPs with different length and hydrophobicity were chosen for this study. The first ELP contains 40 repeats of GVGVP (SEQ ID NO: 4) units (hence referred to as V₄₀), while the other ELP comprised of 80 repeats of GZGVP (SEQ ID NO: 5) with the composition of Z=80% V and 20% A, referred to as (V/A)₈₀. This ELP also contained 8 lysine residues distributed throughout the sequence.

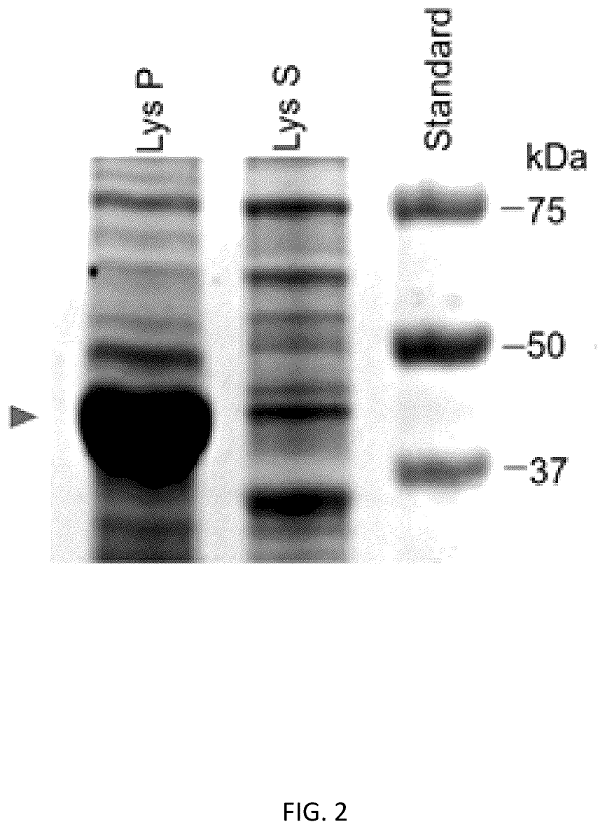

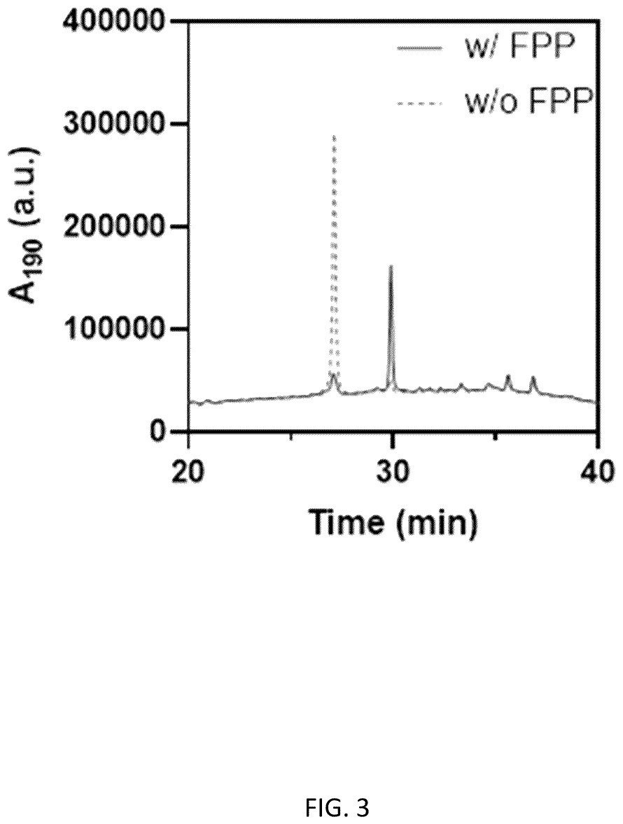

To identify conditions for high-yield heterologous production of the transferase, the pETDuet-1 vector was initially used to co-express the prenyltrasferase subunits in BL21(DE3) strains. This bicistronic vector uses two independent T7/lac promotors to independently produce alpha and beta subunits. Despite the high expression efficiency of this vector, almost all the expressed transferase was found in insoluble inclusion bodies, as seen in FIG. 2. Unfortunately, changes to expression media, temperature, inducer amount, and induction time did not increase the soluble protein production. Since the individual subunits of prenyltransferase tend to aggregate when not bound to their complementary subunits, a translational coupling system was next used to link the production of alpha and beta subunits. In this construct, the stop codon (TAA) of the β-subunit overlaps with the start codon of α-subunit, (TAATG) (SEQ ID NO:6). This translationally coupled system had previously been used to produce the F/GG-Tase in E. coli at quantities sufficient for biochemical characterization. It was then verified that this plasmid produces recombinant, soluble prenyl transferase in E. coli, which can lipidate its protein substrate in vitro, as seen in FIG. 3.

The laborious and costly synthesis of FPP hinders the in vitro production of farnesylated proteins at a scale sufficient for various biomedical and material engineering applications. Therefore, it was investigated whether co-expression of protein substrate and the transferase can be used to recombinantly produce desired farnesylated proteins in a one-pot expression-lipidation system as seen in FIG. 4b. HPLC was used to determine the ratio of lipidated and nonlipidated ELP-CaaX (SEQ ID NO: 1), in the absence or presence of prenyl transferase. As shown in this figure, when ELP-CaaX (SEQ ID NO: 1) is expressed alone, the protein elutes at 9.7 min (the peak at 9.8 min, marked with an arrow, is from the disulf-de-bonded dimer). When ELP-CaaX (SEQ ID NO: 1) is expressed in the presence of prenyl transferases, the intensity of unmodified peak was reduced and another peak with a longer retention time (15.2 min) was observed on the chromatogram. The increased retention time is consistent with the increased hydrophobicity of the lipidated protein. Additional molecular characterization (vide infra for details) was consistent with the assignment of this peak to farnesylated product.

Having established that one-pot recombinant expression of farnesyl-modified proteins is possible, the conditions to maximize yields of farnesylated proteins was systematically characterized. The efficiency of farnesylation in E. coli is influenced by concentration of the available intracellular pool of FPP, the binding constant of FPP to each prenyl transferase, and the rate of lipid transfer to the protein of interest, which is influenced by accessibility of the CaaX motif (SEQ ID NO: 1), and the identity of “X”-residue. Since a priori estimation of these variables is difficult, it was determined the amount of farnesylated proteins using a 2×2 factorial design with prenyl transferase (FTase and GGTase-I) and the identity of X-residue (Ser and Leu). FIG. 4c shows the production yield of (V/A)₈₀-Fr, in mg/L of culture, in these four combinations. In this in vivo biosynthetic system, FTase was able to farnesylate proteins fused to both CVLS (SEQ ID NO: 2) and CVLL (SEQ ID NO: 3) (its canonical and noncanonical substrates) with similar efficiency. On the other hand, while FPP is not the canonical lipid donor for GGTase-I, this enzyme had the highest production yield for farnesylation of (V/A)₈₀-CVLL (SEQ ID NO: 3) but did not modify (V/A)₈₀-CVLS (SEQ ID NO: 2). One-way ANOVA showed a statistically significant difference between the groups (F(3,8)=28.73, p=0.0001). Tukey's HSD Test for multiple comparisons found that the yield of (V/A)₈₀-Fr produced by GGTase-I/CVLL (SEQ ID NO: 3) was significantly higher than other combinations of enzyme/substrate, i.e., GGTase-I/CVLS (SEQ ID NO: 2) (p<0.0001, 95% C.I.=[1.9 to 3.6]; FTase/CVLS (SEQ ID NO: 2) (p=0.0005, 95% C.I.=[1.0 to 2.5]), and FTase/CVLL (SEQ ID NO: 3) (p=0.01, 95% C.I.=[0.4 to 2.2]). GGTase-I/CVLL (SEQ ID NO: 3) was then used for scaled-up production of farnesylated protein in a conventional biosynthetic platform.

Large scale protein expression and purification of both V₄₀-Fr and (V/A)₈₀-Fr (and their nonlipidated controls) were conducted according to protocols described in the supporting information. Briefly, plasmids encoding the α- and β-subunits of GGTase-I and substrate protein fused to CVLL (SEQ ID NO: 3) were co-transformed to BL21 (DE3) E. coli strains. The bacteria were cultivated at 37° C. to OD₆₀₀˜0.8. At this point, the temperature was reduced to 28° C. and the expression culture was supplemented with ZnSO₄ (0.5 mM, metal cofactor required for GGTase-I), and the co-expression of GGTase-I and polypeptide fused to the enzyme recognition sequence was induced by IPTG (1 mM). After 18 h, cells were harvested by centrifugation. The ELPs were isolated by adapting a method recently developed by Thompson and coworkers to purify post-translationally lipidated ELPs. This approach uses a combination of organic (non) solvents to lyse the cells and to selectively isolate the ELP and hybrid biopolymers (as shown in this paper) from the complex mixture of cellular proteins.

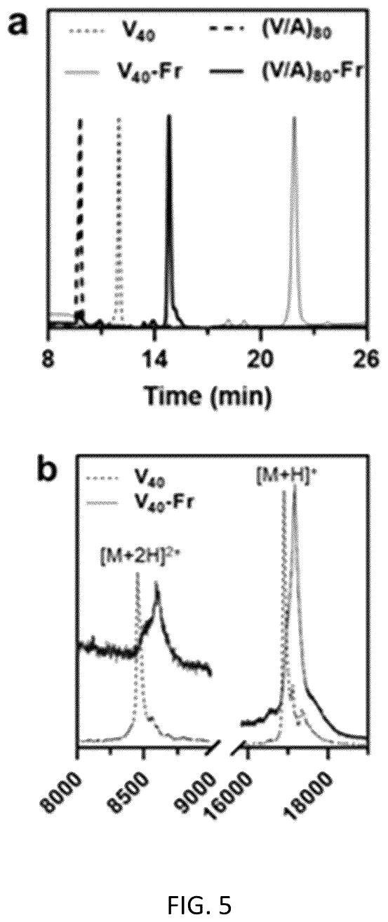

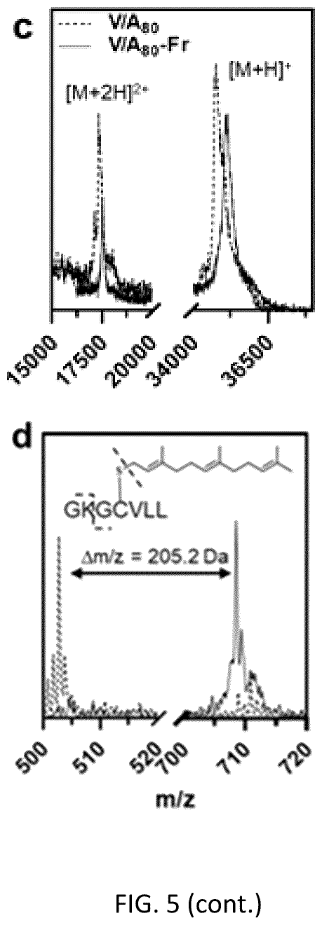

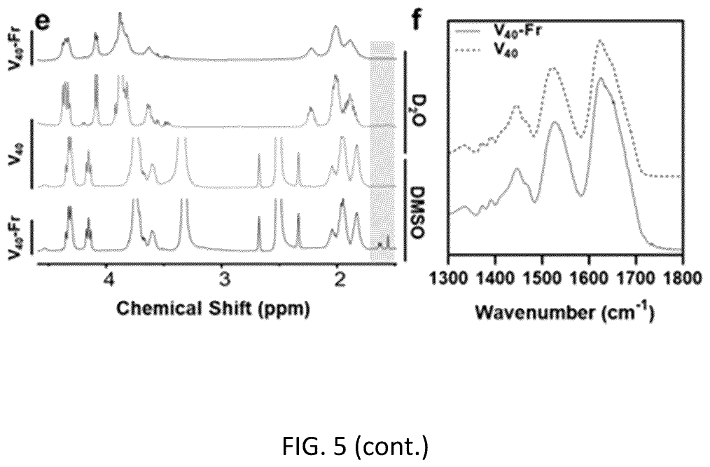

After purification, a combination of HPLC, MALDI-TOF MS, and NMR was used to verify the identity and purity of constructs, and to establish that only one farnesyl group is added to the protein at the cysteine residue located in the C-terminal CaaX motif (SEQ ID NO: 1), as seen in FIG. 5. As shown in FIG. 5a, analytical RP-HPLC confirms the purity of each construct and provides a measure of their hydrophobicity based on their retention time. As expected, farnesylated proteins had longer retention times compared to their unmodified analogs, which is consistent with their increased hydrophobicity. The unmodified V₄₀-CVLL (SEQ ID NO: 3) was eluted at 12.0 min (FIG. 5a, black dotted curve), while the V₄₀-Fr was eluted at 21.9 min (solid curve); The unmodified (V/A)₈₀-CVLL (SEQ ID NO: 3) was eluted at 9.8 min (black dashed curve), while (V/A)₈₀-Fr eluted at 14.8 min (thicker solid curve).

For both ELPs, the molecular ion peak corresponding to lipidated protein was shifted by m/z=+205.2 Da, consistent with the addition of a farnesyl motif (and removal of a hydrogen atom from the thiol). In each case, the doubly charged ion [M+2H]²⁺ was detected, as seen in FIG. 5b,c. To identify the location of the modification, (V/A)₈₀-Fr was digested with trypsin and the resulting peptide fragments were analyzed with MALDI-TOF-MS. Trypsin cleaves the peptide backbone after positively charged amino acids, such as lysine. There are eight lysines in (V/A)₈₀, which are distributed throughout the sequence of ELP with one located before the CVLL (SEQ ID NO: 3) recognition sequence. As shown in FIG. 5d, we observed a peak at m/z=708.5 Da, which was assigned to S-farnesylated GCVL (SEQ ID NO: 7). This peak was not present in the non-lipidated control, which instead contained a peak at m/z of 502.8 Da, corresponding to unmodified GCVLL (SEQ ID NO: 8) peptide.

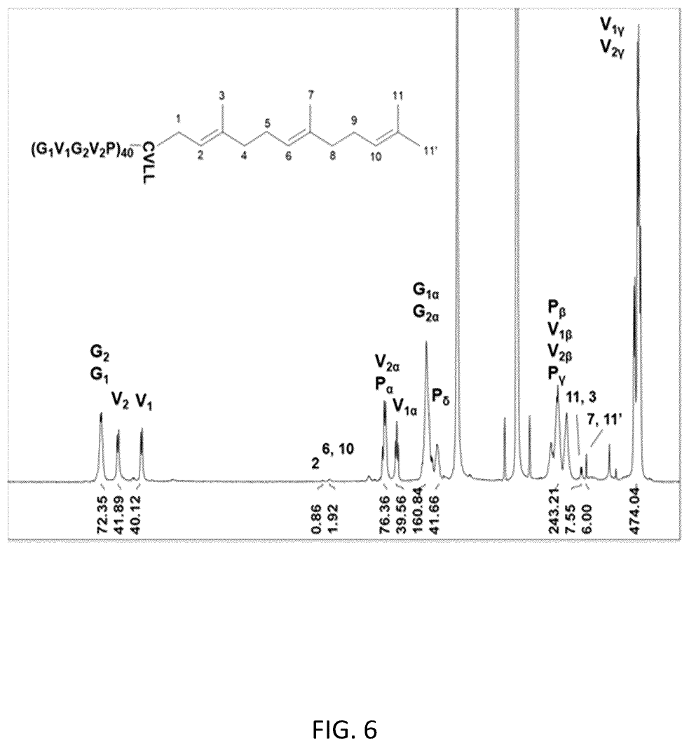

Because V₄₀-Fr lacked a trypsin digestion site near the farnesylation site, 1H NMR spectroscopy was used to confirm its farnesylation. The spectra of V₄₀ and V₄₀-Fr in D₂O is shown in FIG. 5e. Even though the sequence of ELPs is highly repetitive, their sequence-defined and mono-dispersed nature often results in sharp NMR peak (contrary to the spectra of synthetic polymers), FIG. 5e, cyan spectra). However, the spectra of V₄₀-Fr in D₂O exhibited broad peaks (FIG. 5e, purple spectra), and lacked signals corresponding to the farnesyl group. It was hypothesized that the broad peaks are due to the lipid-induced oligomerization of the proteins, which reduces molecular tumbling and both longitudinal and transverse relaxation rates of micellar assemblies. The spectra of V₄₀ and V₄₀-Fr in deuterated DMSO was therefore collected as this organic solvent can disrupt the hydrophobic core of assemblies, resulting in the formation of freely diffusing protein chains (i.e., soluble unimers). As shown in the FIG. 5e, spectra collected in DMSO confirms this hypothesis as both V₄₀ and V₄₀-Fr gave rise to sharp peaks (green and maroon spectra). Moreover, signals corresponding to the farnesyl's allylic methyl groups were clearly visible in 1.5-1.7 ppm and their integration matched the theoretical 1 farnesyl group per protein chain, as seen in FIG. 6.

Finally, FT-IR was used to see if lipidation perturbs the structure of ELPs by comparing the amide absorption bands of unmodified and farnesylated V₄₀ (FIG. 5f). In each case, the FT-IR absorption band maxima was consistent with the presence of large, disordered protein domains, consistent with absence of significant secondary structure after lipidation. The weak absorption of farnesyl C═C or C—H stretches in the FT-IR was unable to be detected, which were likely buried by the strong signals originating from the polypeptide structure.

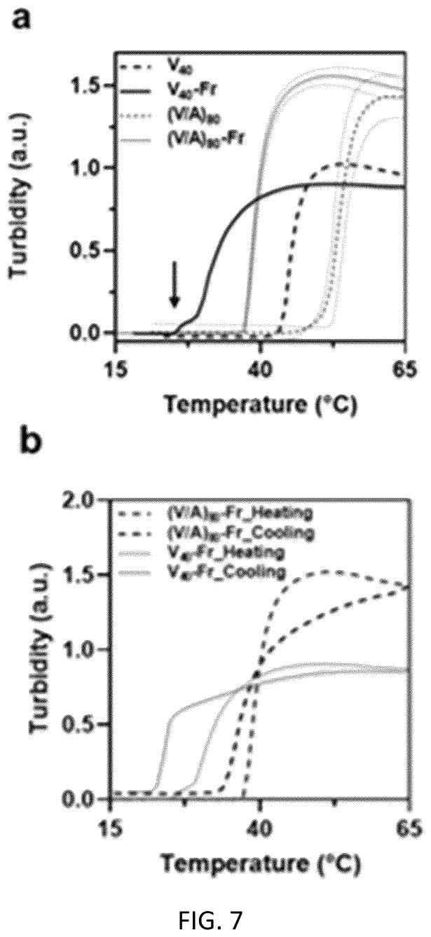

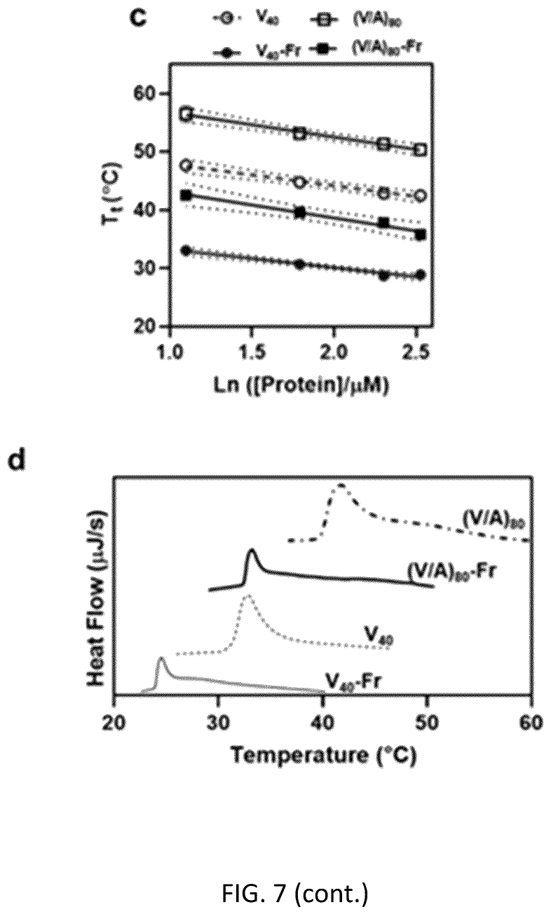

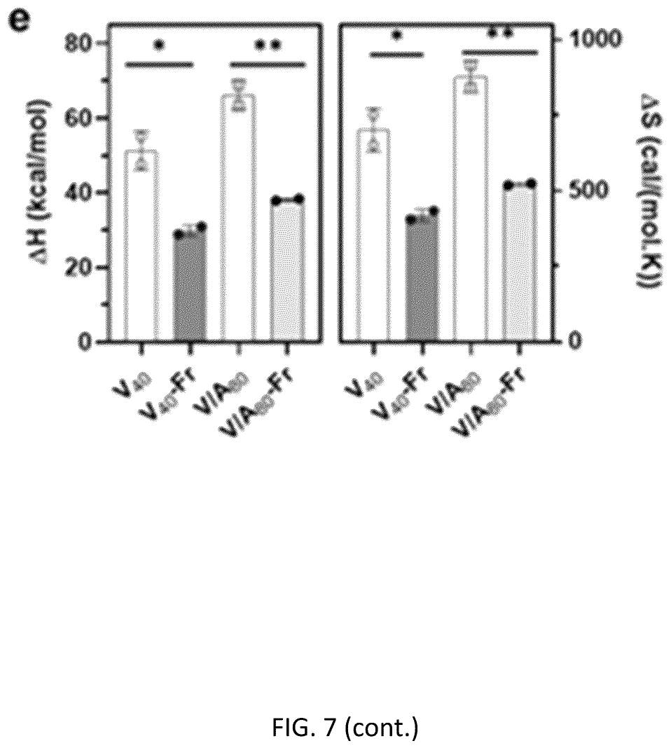

Biopolymers with programmable thermo-response (such as ELPs) are attractive materials for biomedical applications because temperature can be increased locally as a therapeutic modality while causing minimal damage to healthy tissues. While the effect of amino acid mutations on the liquid-liquid phase separation of proteins is under intense investigation, our understanding of the effect of lipidation on the phase-boundaries remains incomplete. Given the importance of thermo-response in biomedical applications, we used complementary techniques of turbidimetry and DSC to quantify the effect of farnesylation on the temperature-triggered phase separation of elastin-based proteins. FIG. 7a shows the turbidity of the solution of nonlipidated and farnesylated ELPs (6 μM in PBS) as a function of temperature. All proteins, except for V₄₀-Fr, show a rapid and one-step transition, characterized by the rapid increase in the turbidity of solution as the temperature is increased above the LCST transition temperature (Tₜ). The turbidity of the solution of V₄₀-Fr is initially increased modestly around 27° C. (marked with an arrow in FIG. 7a, blue solid curve), which is followed by a sharp increase when T>29° C. The differences in the cumulative turbidity of the solutions between V₄₀ and (V/A)₈₀ constructs are due to differences in weight fraction of each polypeptide in solution, (i.e., (V/A)₈₀ has approximately double the molecular weight of V₄₀).

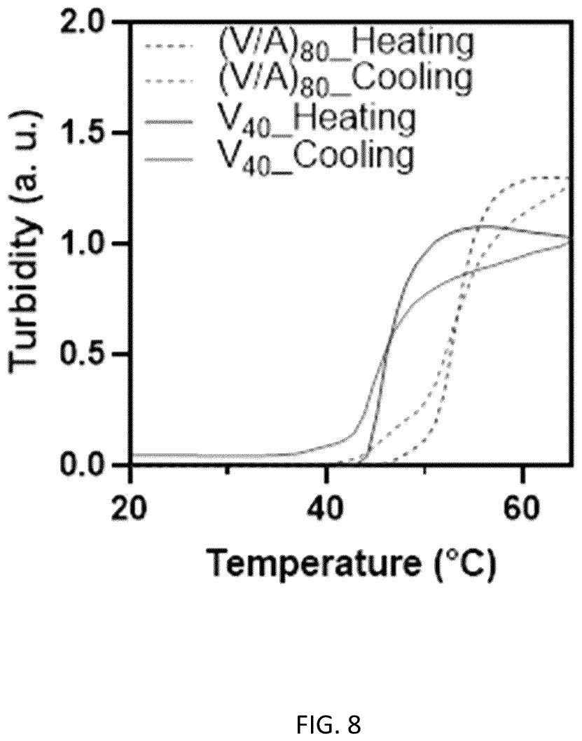

To determine whether the phase separation of farnesyl-modified proteins is reversible, the turbidity of the solution was monitored as the temperature was reduced from 65 to 15° C. with the cooling rate of 1° C./min (blue curves). As shown in FIG. 7c (and FIG. 8), the phase separation of all constructs was completely reversible as the turbidity of the solution reached its initial values before heating, albeit V₄₀-Fr exhibited slight hysteresis as evident by the area between the dashed heating and cooling curves. This kinetic effect is likely due to the combination of farnesyl-mediated oligomerization, and increased hydrophobicity of V₄₀ (compared to (V/A)₈₀), which strengthens ELP-ELP interactions after coacervation, and increases the barrier for dissolution of coacervates.

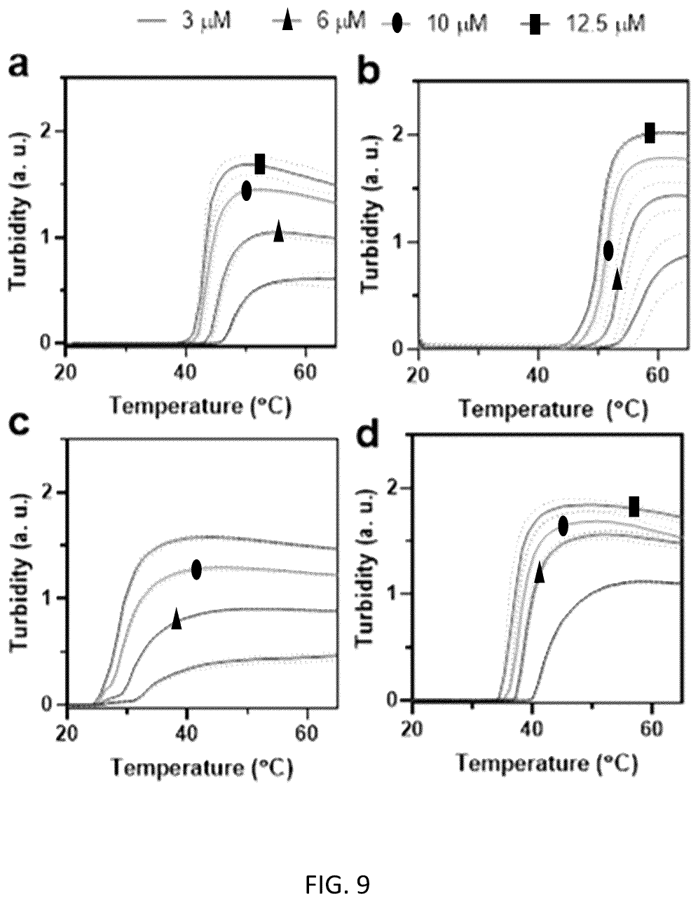

The effect of farnesylation on the concentration dependence of Tₜ was also evaluated for both constructs. Quantifying this behavior is critical for the development of models that can reliably predict the behavior of farnesylated constructs in biomedical applications in which the concentration of protein changes as a function of time, (e.g., thermally triggered drug depots, intravenous administrations, or hyperthermia-based targeting and treatment of cancer). Turbidimetry was used to measure the transition temperature for unmodified and farnesylated proteins at various concentrations (3, 6, 10, 12.5 μM in PBS), as seen in FIG. 9. Transition temperature was defined as the inflection point (i.e., the maximum of the first derivative, dAbs/dT) and was plotted against the natural log of protein concentration to develop a temperature-composition portrait (FIG. 7c). For all constructs within this concentration range, the transition temperature changed linearly with the natural log of the protein concentration (see Table 3 below). Farnesylation did not change the concentration dependence of Tₜ (i.e., the slope of the line) as no statistically significant difference between slopes of the nonlipidated and farnesylated proteins were observed (ANCOVA for V₄₀ constructs: F(1,8)=1.37 p=0.3; for (V/A)₈₀ constructs: F(1,8)=0.07, p=0.8). On the other hand, farnesylation reduced the Y-intercept (i.e., Tₜ) by 15.2° C. for V₄₀ and 13.8° C. for (V/A)₈₀, a statistically significant difference between nonlipidated and farnesyl-modified proteins, ANCOVA for V₄₀ constructs: F(1,9)=2705, p<0.0001; for (V/A)₈₀ constructs F(1,9)=1107, p<0.0001.

To investigate how farnesylation modulates the thermodynamics of liquid-liquid phase separation, we used DSC to measure the total heat of phase separation. FIG. 7d presents results from DSC measurements performed on nonlipidated and farnesyl-modified V₄₀ and (V/A)₈₀ constructs. The LLPS of ELP is distinguished by an endothermic peak, and the area under this peak provides an estimate of the ΔH of the phase separation process. As shown in FIG. 7d (and Table 4 below), farnesylation reduces the area of the peak and it also notably increases its asymmetry. This observation suggests that farnesylation not only changes the thermodynamics of phase separation, but it may also change processes such as nucleation, coalescence or ripening of coacervates. Given the positive ΔH of phase separation for both lipidated and nonlipidated proteins, the driving force for phase separation is the favorable and positive entropy (ΔS), due to the interactions of hydrophobic residues and the release of “frozen” water molecules that constitute the hydration shell of ELPs. Nonetheless, DSC also revealed that farnesylation lowers the enthalpy (ΔH) of coacervation for both constructs (two-tailed unpaired t-test; (V₄₀ VS. V₄₀-Fr): t(2)=5.63, p=0.03; (V/A)₈₀ vs. (V/A)₈₀-Fr: t(2)=10.09, p=0.0097) , and thus have a stabilizing effect (−22-28 kcal/mol) on the coacervates. This is likely through favorable van der Waals interactions between farnesyl groups (i.e., the ΔH of micellization<0) or because it changes the interactions of ELP chains within the micelle.

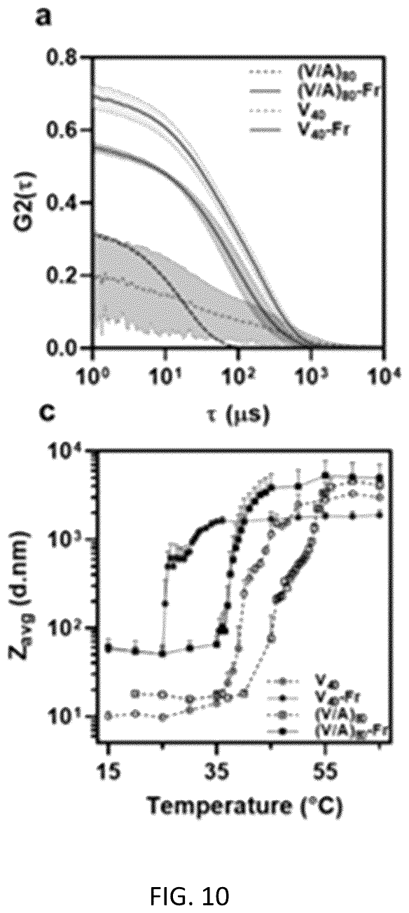

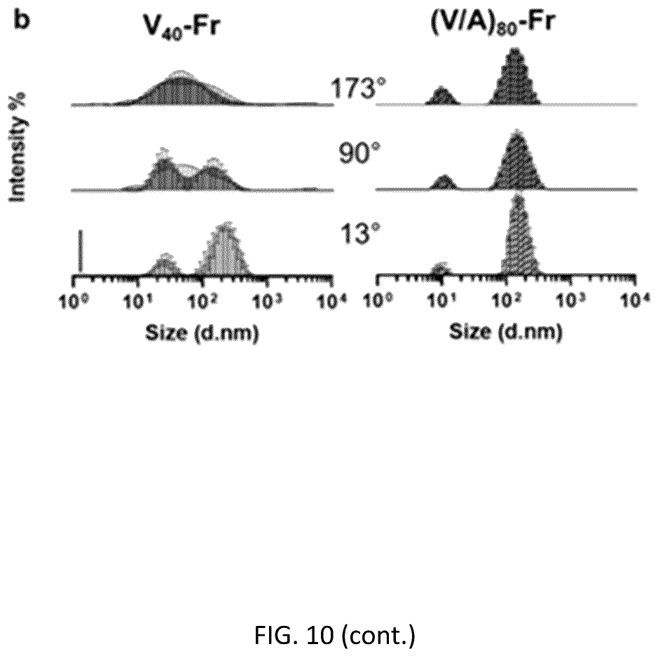

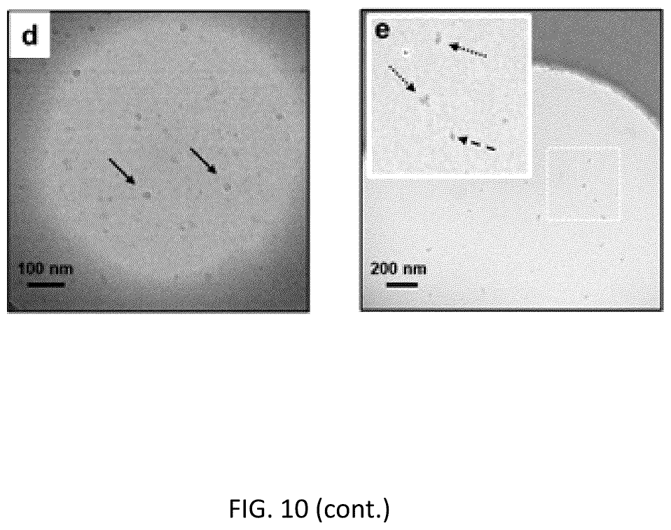

As discussed earlier, the broad peaks in the NMR (FIG. 5e) and reduced enthalpy and entropy of phase separation (FIG. 7e) suggest that farnesylated constructs may self-assemble. Therefore, the effect of farnesylation on the supramolecular assembly of these constructs was investigated using multi-angle dynamic light scattering (MADLS) and cryogenic transmission electron microscopy (cryo-TEM) to characterize these assemblies at higher resolutions. FIG. 10a shows the autocorrelation functions (ACF) derived from analysis of raw scattering data collected at 173°. The ACFs of unmodified ELPs are characterized by a fast decay and low Y-intercept (<0.5). On the other hand, the ACFs of farnesylated proteins exhibited a slower decay (increase in the X-intercept) and higher Y-intercept (>0.5), which is consistent with the formation of larger assemblies with stronger scattering profiles.

The visual inspection of ACFs for farnesyl-modified proteins also shows that the exponential decay profile is not monotonic, which likely is due to equilibrium between assemblies of different size. The bimodal size-intensity distributions at different angles (13°, 90°, and 173°) shown in FIG. 10b are consistent with this conjecture. Because the larger particles scatter light more strongly at smaller angles, the MADLS profile can be used to improve the resolution between the two populations (c.f., V₄₀-Fr distributions at 173° vs. 13°). This dynamic equilibrium is distinct from the behavior of supramolecular assemblies formed by proteins modified with fatty acids or sterols, which formed static assemblies with shapes and sizes governed by the molecular composition of LP.

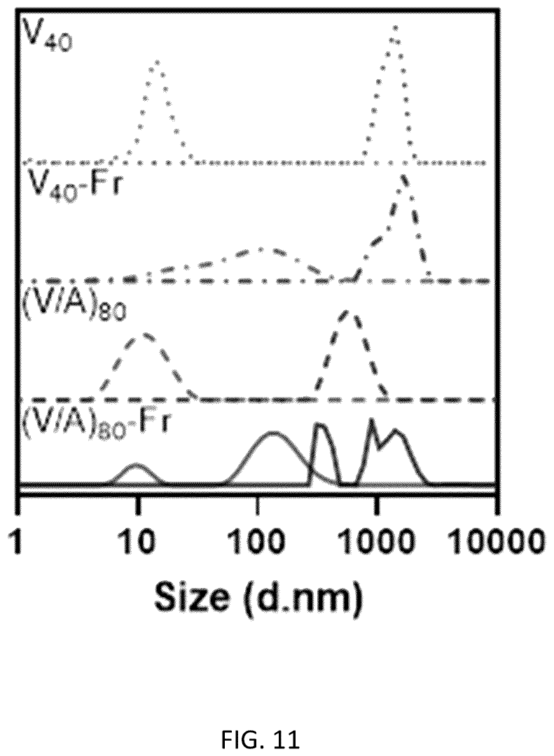

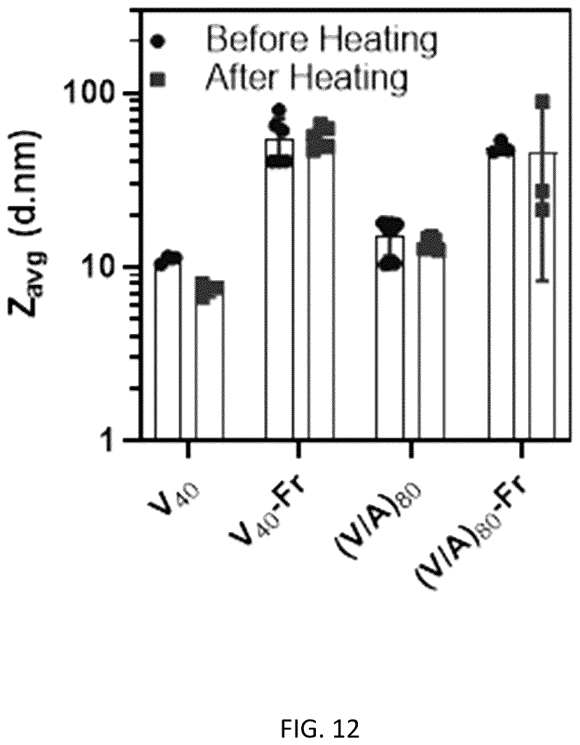

To determine the effect of temperature on the properties of these assemblies, DLS was conducted from 15-65° C. Initially, temperature steps were set at 5° C., but smaller temperature steps (0.5-1° C.) were applied within Tₜ (turbidimetry)±5° C. The ACF at each temperature was analyzed using the method of cumulants to calculate the average hydrodynamic diameter of proteins at each temperature (FIG. 10c, Table 5 below). In each case, the hydrodynamic radius increased by 1 to 2 orders of magnitude T>Tₜ, which is consistent with the formation of mesoscale coacervates at higher temperatures, as seen in FIG. 11. The behavior of V₄₀-Fr constructs was slightly different; the temperature-triggered increase in the hydrodynamic radius appeared to occur within two distinct stages with the first increase at 25-28° C. followed by a secondary increase at T>28° C., a behavior consistent with the turbidimetry results. Moreover, DLS also confirmed that the temperature-triggered phase-separation of both unmodified and lipidated proteins is reversible (FIG. 12).

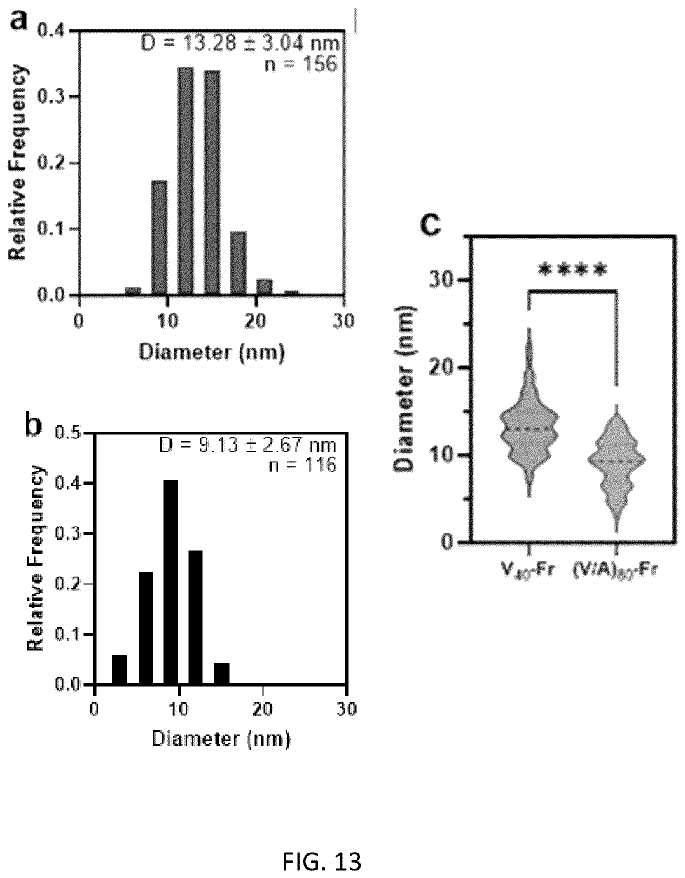

Cryo-TEM was used to image the assemblies of farnesylated proteins at 20° C. (below their transition temperature). Intriguingly, noticeable differences was observed in the nanostructure of V₄₀-Fr and (V/A)₈₀-Fr, even though DLS yielded similar Z_{avg} for both constructs. As shown in FIG. 10d (see FIG. 13 for the histogram depicting the size distributions), V₄₀-Fr formed spherical nanoparticles with average size of 13.3±3.0 nm (FIG. 10d, solid arrows) which exhibited relatively uniform contrast (i.e., hydration level). On the other hand, the cores of (V/A)₈₀ assemblies were noticeably smaller 9.1±2.7 nm (FIG. 10e, dashed arrow), but supra-particles were observed that appeared to form from the association of these smaller particles (FIG. 10e, dotted arrows). Note. However. that cryo-TEM visualizes assemblies based on differences in the hydration. Since ELP chains are hydrated below their transition temperature, cryo-TEM may only visualize the hydrophobic core of the assemblies.

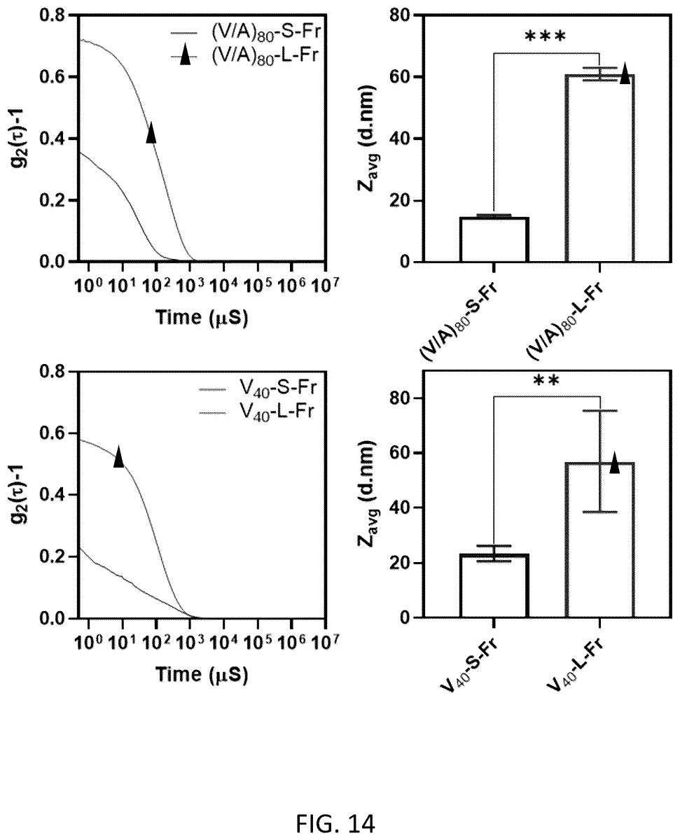

Referring to FIG. 14, the low intercept and decay time for proteins with a -CVLS (SEQ ID NO: 2) construct (FIGS. 14a and c) indicate lack of self-assembly. The size was different for -CVLL (SEQ ID NO: 3) and -CVLS (SEQ ID NO: 2) constructs for both (V/A)₈₀-S-Fr (t(2)=34.18, p=0.0009) and V₄₀-S-Fr (t(2)=20.82, p=0.0023). This data shows that non-canonical farnesylation signals (with hydrophobic X-residue) are necessary to form such nanoparticles from farnesylated proteins.

By converging synthetic biology with materials science, the present invention provides recombinant platforms to produce farnesylated proteins with programmable assembly and temperature-dependent characteristics. Although in vitro and in lysate prenylation of proteins has been previously demonstrated, the laborious and expensive synthesis of prenyl donors limits the scalability of these methods. Consequently, they are typically used to produce small quantities (a few μg) of naturally occurring farnesylated proteins for biochemical characterization or chemoenzymatic labeling of proteins with bio-orthogonal handles. The method 10 of the present invention enables scalable production of farnesylated proteins outside of the biological context (i.e., with artificial proteins and/or noncanonical lipidation sites) for biomaterial and biomedical applications. The present invention also captures the differences between closely related prenyl transferases for efficient modification of proteins in these recombinant platforms. Seminal biochemical studies had previously established that GGTase-I can accept both FPP and GGPP as the lipid donor, and the identity of the X-residue in CaaX (SEQ ID NO: 1) box alters the substrate-preference of transferase. Nonetheless, the present invention reveals that the concentration and availability of prenyl donors inside a microbial factory are different from the conditions often used to characterize the biochemistry of enzymes using large excess of lipid or peptide substrate. The present invention also demonstrates that the identity of the X-residue determines whether farnesylated proteins can self-assemble into recombinant nanoparticles (if X is hydrophobic amino acids such as valine) or not (when X is hydrophilic amino acids such as serine), FIG. 14. Such nanoparticles can be used to encapsulate hydrophobic therapeutics (e.g., doxorubicin and paclitaxel) or display biologically active peptides such as agonists/antagonists of class B G-protein-coupled receptors (e.g., exendin which activates GLP-1R for insulin secretion or calcitonin which activates calcitonin receptor for the maintenance of calcium homeostasis in bone formation).

By combining biophysical and soft-matter characterization techniques, it has been shown that farnesylation alters the thermo-response, phase separation, and assembly of ELPs used as model protein system. These effects are consistent with the physicochemistry of the farnesyl group, as this hydrophobic lipid increases the unfavorable interactions of ELP with water and modulates the energetics of the phase separation via the supramolecular oligomerization of farnesyl-modified proteins. Dynamic light scattering and cryo-TEM show that the farnesylated assemblies likely exist in a dynamic equilibrium between unimers and assembled structures. The chemical structure of farnesyl, which contains three unsaturated bonds, is likely responsible for the reduced hydrophobicity and increased dynamics of micellar assemblies. The differences observed between the properties of farnesylated proteins vs. proteins modified with saturated fatty acid or sterols strongly suggest that the physicochemical properties of the lipid can be used as a design principle to control the biophysical and material properties of this class of hybrid materials.

Recent biochemical studies have greatly expanded the size of potential prenylated proteome beyond the classic CaaX motifs (SEQ ID NO: 1) to other motifs such as C(x)3X and Cxx. Nonetheless, the correlation between the sequence of these lipidation sites and the assembly or localization of prenylated proteins remains less clear. The present invention sets the stage for future studies to reveal the interplay of prenylation sites or the effect of post-prenylation processing steps on the emergent properties of farnesylated proteins. Moreover, the strategy of the present invention can be applied to the biosynthesis of geranylgeranylated proteins by metabolically engineering E. coli to increase the intracellular GGPP. These studies are underway in our laboratories and will be reported in due course.

### Example 1

Cloning. Genes encoding α and β subunits of FTase and GGTase-I (from Rattus norvegicus) as well as their canonical substrate peptides, CVLS (SEQ ID NO: 2) and CVLL (SEQ ID NO: 3) were constructed using standard molecular biology techniques. All cloning steps were conducted using E. coli EB5α strain. The identity of each gene was confirmed by Sanger sequencing. After verifications, plasmids were co-transformed to E. coli BL21(DE3) for protein expression. See Table 1 below for additional details.

TABLE 1
Plasmids used for expressing the proteins

| | Vectors used for | |
|---|---|---|
| Constructs | expression | Relevant features of the vector |
| V₄₀ | pJMD^{a} | Kan^{r}, pBR322 Ori, monocistronic T7 |
| | | promotors |
| | pACYCDuet-1 | Cm^{r}, p15A Ori, bicistronic T7 promotors |
| (V/A)₈₀ | pJMD | Kan', pBR322 Ori, monocistronic T7 |
| | | promotor |
| | pACYCDuet-1 | Cm^{r}, p15A Ori, bicistronic T7 promotor |
| GGtase-I | pET23a | Amp^{r}, pBR322 Ori, monocistronic T7 |
| | | promotor |
| FTase-I | pET23a | Amp^{r}, pBR322 Ori, monocistronic T7 |
| | | promotor |

### Expression of Non-Lipidated Constructs

A bacterial colony was used to inoculate a flask containing 50 mL of sterile 2×YT medium supplemented with kanamycin (45 mg/mL). After overnight growth in a shaking incubator (37° C., 200 rpm, 16 h), 4 mL of this culture was used to inoculate 1 L of 2×YT media. The bacteria were grown in an orbital shaker incubator at 37° C. at 200 rpm. The expression was induced by adding isopropyl β-D-1-thiogalactopyranoside (IPTG) to a final concentration of 1 mM when the optical density reached 1.5. After 18 hours, cells were harvested by centrifugation (3745×g, 4° C. for 30 min). The bacterial pellet was resuspended in phosphate buffer saline (PBS, pH 7.4, 10 mL/L of expression culture). The cells were stored at −80° C. until purification.

### One-Pot Expression-Lipidation of Farnesylated-Proteins

A 50 mL of sterile 2×YT medium, supplemented with ampicillin (100 mg/mL) and chloramphenicol (25 mg/mL), was inoculated with a bacterial colony and incubated in an orbital shaker (37° C., 200 rpm) to OD600 of 1.5. 4 mL of this media was used to inoculate each one liter of 2×YT medium. The bacteria were grown in an orbital shaker incubator at 37° C. at 200 rpm. After reaching to OD600 of 0.8, the temperature was reduced to 28° C. and the expression was induced by adding IPTG and ZnSO₄ to the final concentrations of 1 mM and 0.5 mM, respectively. After continuing the expression at 28° C. for 18 h, the cells were harvested by centrifugation (3745×g, 4° C., 30 min). The bacterial pellet was resuspended in PBS (pH 7.4, 5 mL/L of expression culture).

### Protein Purification

Proteins were purified by adapting a recently reported method for rapid purification of ELPs for isolation of post-translationally lipidated proteins. Briefly, the cell pellets were incubated with isopropanol, which lyses the cells and precipitates most endogenous proteins and nucleic acids. After separation of insoluble debris, the expressed ELPs are precipitated by adding a non-solvent, acetonitrile to the final volume of 70% (v/v). The protein pellet was resuspended in 50% (v/v) ethanol in water and purified by preparative RP-HPLC to ensure >95% purify for characterization studies.

### RP-HPLC

Preparative and analytical RP-HPLC were performed on a Shimadzu instrument equipped with a photodiode array detector on C18 columns (Phenomenex Jupiter® 5 μm C18 300 Å, 250×4.6 mm and 250×10 mm). A mobile phase consisting of gradient of acetonitrile and water (supplemented with 0.1% TFA) was used to elute the proteins, as seen in Table 2 below:

TABLE 2
The gradient mobile phase composition of analytical HPLC

| Time (min) | % (CH₃CN + 0.1% TFA) |
|---|---|
| 0 | 0 |
| 5 | 40 |
| 12 | 50 |
| 22 | 53 |
| 23 | 90 |
| 28 | 90 |

### Turbidimetry Assay

The thermal response of proteins was analyzed using a Cary 100 UV-Vis Spectro-photometer (Agilent, Santa Clara, CA) equipped with a Peltier temperature controller. The absorbance of protein solutions at 350 nm were continuously monitored between 15-65° C. while heating/cooling the solution at the rate of 1° C./min.

### Matrix-Assisted Laser Desorption/Ionization, Time-of-Flight Mass Spectrometry (MALDI-TOF-MS)

The molecular weight of proteins was determined using MALDI-TOF-MS, conducted on a Bruker Microflex® LRF instrument. To determine the location of farnesyl group, the proteins were digested with trypsin, and the peptide fragments were analyzed using MALDI-TOF-MS.

### Multi-Angle Dynamic Light Scattering (MADLS)

MADLS was performed using Zetasizer Ultra (Malvern Instruments, UK) at the scattering angles of 13°, 90°, and 173°. Protein solutions (6 μM in PBS) were filtered into a DLS cuvette and analyzed at 15-65° C. Measurements were performed in triplicate after incubating the samples for 3 min at each temperature. Scattering autocorrelation functions were analyzed with Zetasizer software using the cumulant method to derive average hydrodynamic radius (Zavg) and polydispersity index. The intensity-size distributions are calculated using CONTIN method.

### Cryo-Transmission Electron Microscopy (Cryo-TEM)

Protein solutions were applied to a freshly plasma cleaned grid (Pelco easiGlow, negative polarity, 45 s, 30 mA) and plunged frozen in liquid ethane. Grids were stored under liquid nitrogen until they were imaged on a Tecnai BioTwin 120 kV transmission electron microscope, operated at LN2 temperature. Samples were imaged under low-dose conditions using a Gatan 626 or a Gatan 910 holders, cooled to LN2 temperature. Images were collected on a Gatan SC1000A CCD-camera. TEM images were analyzed using ImageJ.

### Differential Scanning Calorimetry (DSC)

NanoDSC (TA instruments, New Castle) was used to quantify the enthalpy of phase-separation by measuring the excess heat capacity of the protein solution (against PBS reference) while heating the sample 10 to 65° C. at a rate of 1° C./min.

### Attenuated Total Reflectance Fourier-Transform Infrared Spectroscopy (ATR-FTIR)

The FT-IR absorption spectra were collected using Thermo Scientific Nicolet iS5 FT-IR Spectrometer with iD7 attenuated total reflectance accessory by sandwiching the lyophilized proteins directly over the crystal. The spectral resolution was set to 4 cm-1 and each spectrum were obtained with 128 scans.

### Proton Nuclear Magnetic Resonance (1H NMR)

1H NMR spectra were recorded with Bruker Avance III HD 400 MHz. The samples were prepared by dissolving lyophilized proteins into deuterium oxide or dimethyl sulfoxide-d6 at the concentration of 1.67 mg/mL. The proton NMR spectra were collected at 25° C.

The pACYCDuet-I vector was purchased from EMD Millipore (Billerica, MA). The chemically competent Eb5α and BL21(DE3) cells, restriction enzymes, ligase, and corresponding buffers, as well as DNA extraction and purification kits, were purchased from New England Biolabs (Ipswich, MA). Isopropyl β-D-1-thiogalactopyranoside (IPTG) was purchased from A. G. Scientific (San Diego, CA). Apomyoglobin, adrenocorticotropic hormone (ACTH), sinapinic acid, alpha-cyano-4-hydroxycinnamic acid, zinc sulfate, and trifluoroacetic acid (TFA) were purchased from Sigma-Aldrich (St. Louis, MO). High-performance liquid chromatography-(HPLC) grade acetonitrile, SnakeSkin™ dialysis tubing with 3.5K nominal molecular weight cut off (MWCO), mass spectroscopy grade Pierce™ trypsin protease, tryptone, yeast extract, sodium chloride, ampicillin, kanamycin, chloramphenicol, phosphate buffer saline (PBS), DMSO, isopropanol, acetonitrile, and ethanol were purchased from Thermo Fisher Scientific (Rockford, IL). Mini-PROTEAN® TGX Stain-Free™ Precast Gels, Precision Plus Protein™ All Blue Pre-stained Protein Standard, and Precision Plus Protein™ Unstained Protein Standards were purchased from Bio-Rad Laboratories, Inc. (Hercules, CA). Deionized water was obtained from a Milli-Q® system (Millipore SAS, France). Simply Blue™ SafeStain was purchased from Novex (Van Allen Way Carlsbad, CA). All chemicals were used as received without further purification.

The sense and antisense oligonucleotides encoding for the canonical peptide substrate of FTase (CVLS) (SEQ ID NO: 2) and GGTase-I (CVLS) (SEQ ID NO: 2) were purchased from IDT DNA. The 5′-ends of single-stranded DNAs were phosphorylated, after which the complementary oligos were thermally annealed. The double-stranded DNA was then cloned into a modified pET24a(+) vector, referred to as pJMD5, which had been double digested with BseRI and BamHI. Recursive directional ligation by plasmid reconstruction was used to fuse the peptide substrates to the C-termini of V₄₀ and (V/A)₈₀ genes.¹ After sequence verification, the ELP-CVLS (SEQ ID NO: 2) or ELP-CVLL (SEQ ID NO: 3) were cloned into pACYCDuet-I using NdeI and XhoI restriction enzymes. Genes encoding for a and b subunits of FTase-I and GGTase-I were ordered as gene fragments from IDT DNA and were cloned into pETDuet-1 using Gibson assembly. The beta subunit was cloned into MCS1 between NcoI and EcoRI sites, and the alpha subunit was cloned into MCS2 between Nde and XhoI. The construction of vectors used for translational coupling of a and b subunits has been reported previously.

V₄₀
(SEQ ID NO: 9)
(M)GVGVPGVGVPGVGVPGVGVPGVGVPGVGVPGVGVPGVGVPGVGVP
GVGVPGVGVPGVGVPGVGVPGVGVPGVGVPGVGVPGVGVPGVGVPGVGV
PGVGVPGVGVPGVGVPGVGVPGVGVPGVGVPGVGVPGVGVPGVGVPGVG
VPGVGVPGVGVPGVGVPGVGVPGVGVPGVGVPGVGVPGVGVPGVGVPGV
GVPGVGVPGCVLL.
(V/A)₈₀
(SEQ ID NO: 10)
(M)GVGVPGVGVPGAGVPGVGVPGVGVPGVGVPGVGVPGAGVPGVGVP
GVGVPGGKGVGVPGVGVPGAGVPGVGVPGVGVPGVGVPGVGVPGAGVPG
VGVPGVGVPGGKGVGVPGVGVPGAGVPGVGVPGVGVPGVGVPGVGVPGA
GVPGVGVPGVGVPGGKGVGVPGVGVPGAGVPGVGVPGVGVPGVGVPGVG
VPGAGVPGVGVPGVGVPGGKGVGVPGVGVPGAGVPGVGVPGVGVPGVGV
PGVGVPGAGVPGVGVPGVGVPGGKGVGVPGVGVPGAGVPGVGVPGVGVP
GVGVPGVGVPGAGVPGVGVPGVGVPGGKGVGVPGVGVPGAGVPGVGVPG
VGVPGVGVPGVGVPGAGVPGVGVPGVGVPGGKGVGVPGVGVPGAGVPGV
GVPGVGVPGVGVPGVGVPGAGVPGVGVPGVGVPGGKGCVLL.
α subunit of FTase and GGTase-I
(SEQ ID NO: 11)
(M)AATEGVGESAPGGEPGQPEQPPPPPPPPPAQQPQEEEMAAEAGEAA
ASPMDDGFLSLDSPTYVLYRDRAEWADIDPVPQNDGPSPVVQIIYSEKF
RDVYDYFRAVLQRDERSERAFKLTRDAIELNAANYTVWHFRRVLLRSLQ
KDLQEEMNYIIAIIEEQPKNYQVWHHRRVLVEWLKDPSQELEFIADILN
QDAKNYHAWQHRQWVIQEFRLWDNELQYVDQLLKEDVRNNSVWNQRHFV
ISNTTGYSDRAVLEREVQYTLEMIKLVPHNESAWNYLKGILQDRGLSRY
PNLLNQLLDLQPSHSSPYLIAFLVDIYEDMLENQCDNKEDILNKALELC
EILAKEKDTIRKEYWRYIGRSLQSKHSRESDIPASV.
β subunit of FTase
(SEQ ID NO: 12)
(M)ASSSSFTYYCPPSSSPVWSEPLYSLRPEHARERLQDDSVETVTSIE
QAKVEEKIQEVFSSYKFNHLVPRLVLQREKHFHYLKRGLRQLTDAYECL
DASRPWLCYWILHSLELLDEPIPQIVATDVCQFLELCQSPDGGFGGGPG
QYPHLAPTYAAVNALCIIGTEEAYNVINREKLLQYLYSLKQPDGSFLMH
VGGEVDVRSAYCAASVASLTNIITPDLFEGTAEWIARCQNWEGGIGGVP
GMEAHGGYTFCGLAALVILKKERSLNLKSLLQWVTSRQMRFEGGFQGRC
NKLVDGCYSFWQAGLLPLLHRALHAQGDPALSMSHWMFHQQALQEYILM
CCQCPAGGLLDKPGKSRDFYHTCYCLSGLSIAQHFGSGAMLHDVVMGVP
ENVLQPTHPVYNIGPDKVIQATTHFLQKPVPGFEECEDAVTSDPATDQE
EF

‘QEEF’ at the end is translated from the RBS site that is proceeding the alpha

subunit start codon in the translationally-coupled system (FIG. 4a).

β subunit of GGTase-I
(SEQ ID NO: 13)
(M)AATEDDRLAGSGEGERLDFLRDRHVRFFQRCLQVLPERYSSLETSR
LTIAFFALSGLDMLDSLDVVNKDDIIEWIYSLQVLPTEDRSNLDRCGFR
GSSYLGIPFNPSKNPGTAHPYDSGHIAMTYTGLSCLIILGDDLSRVDKE
ACLAGLRALQLEDGSFCAVPEGSENDMRFVYCASCICYMLNNWSGMDMK
KAISYIRRSMSYDNGLAQGAGLESHGGSTFCGIASLCLMGKLEEVFSEK
ELNRIKRWCIMRQQNGYHGRPNKPVDTCYSFWVGATLKLLKIFQYTNFE
KNRNYILSTQDRLVGGFAKWPDSHPDALHAYFGICGLSLMEESGICKVH
PALNVSTRTSERLRDLHQSWKTKDSKQCSDNVHISSQEEF.

The proteins were purified by optimizing a recently reported extraction method of ELP. The method was optimized to apply for lipidated proteins as well as to reduce the isolation time to 40 min. The cells were pelleted by centrifuging at 21850×g at 4° C. for 5 min. After decanting the supernatant, the cell pellet was resuspended in isopropanol (4 mL/g of the wet pellet). After thorough mixing by vortexing and bath sonication for 5 min, the cells were further mixed with isopropanol by rotating for 5 min at 25° C. The protein was then separated in the supernatant by centrifuging at 15000×g for 5 min at 25° C. The protein was then precipitated by adding acetonitrile to the final composition of 70% (v/v). The solution was then centrifuged at 15000×g for 5 min at 25° C. After discarding the supernatant, the protein pellet was resuspended in 50% (v/v) ethanol in water. The suspension was centrifuged at 15000×g, 25° C. for 5 min to remove any insoluble impurities. The protein was then analyzed by Reverse-phase HPLC (RP-HPLC). After organic extraction, the solution contained both unmodified and farnesylated ELPs. The lipidated product can be separated from the unmodified product by leveraging its lower transition temperature. The farnesylated proteins were then purified by preparative HPLC to ensure purity (>95%) for characterization studies. RP-HPLC was performed with a Shimadzu HPLC system (Phenomenex Jupiter® 5 μm C18 300 Å, LC Column 250×10 mm, solvent A: H₂O+0.1% TFA, solvent B: acetonitrile+0.1% TFA). The percentage of the organic solvent in the mobile phase was increased from 0 to 90% over the course of 23 minutes. After HPLC purification, the organic solvent was removed by dialysis against water using SnakeSkin™ Dialysis Tubing (3500 MWCO, Thermo Scientific) overnight, followed by lyophilization. Lyophilized proteins were stored at −20° C.

Analytical RP-HPLC was performed on a Shimadzu instrument using a Phenomenex Jupiter® 5 μm C18 300 Å, 250×4.6 mm LC Column with a mobile phase consisting of a gradient of acetonitrile in water containing 0.1% trifluoroacetic acid (Table 2) to analyze the proteins. The proteins were analyzed using a photodiode array detector at wavelengths between 190 and 230 nm.

MALDI-TOF-MS was conducted on Bruker Microflex® LRF with a microScout ion source. A saturated solution of sinapinic acid in 50% acetonitrile was used as the matrix. The Samples were prepared by mixing 3 μL of 25 μM protein solutions with 7 μL of the matrix followed by serial dilution. These solutions were plated onto a sample plate and dried at room temperature. Apomyoglobin (M_{w}=16,952.27 Da) was used as standard.

To identify the location of the farnesyl group, (V/A)₈₀-Fr were digested with trypsin, and the peptide fragments were analyzed using MALDI-TOF-MS. To set up the reaction, 9 μL of protein (100 μM) was added to 10 μL of 100 mM ammonium bicarbonate buffer (pH=7.8) in an Eppendorf tube. The reaction was initiated by adding 1 μL trypsin (reconstituted as 5 μg/μL in 50 mM acetic acid) at 37° C. After 3 h, the peptide fragments were analyzed by MALDI-TOF-MS. α-cyano-4-hydroxycinnamic acid was used as the matrix for the analysis of the trypsin-digested peptide fragments. The instrument was calibrated using adrenocorticotropic hormone (M_{w}=2,464.1989 Da).

Temperature-triggered phase separation studies of the proteins were performed with an Agilent UV-Vis Spectrophotometer (Cary100) equipped with a Peltier temperature controller by measuring the absorbance of the solution at 350 nm. Four concentrations (3, 6, 10, and 12.5 μM in PBS) of proteins were analyzed by heating the solution at the rate of 1° C./min from 15 to 65° C. For reversibility studies, the protein solutions were then cooled to 15° C. at the same rate. The Transition temperature (Tₜ) was defined as the inflection point, i.e., the maximum of the first derivative, in the absorbance during the heating cycle. These data were fitted to the following model Tₜ=−m×ln [protein]+T_{c} to derive critical transition temperature (T_{c}) and the concentration dependence of Tₜ (m), summarized in Table 3 below.

TABLE 3
Critical transition temperature (Tc) and the concentration
dependence of Tt derived from turbidity plots.

| Construct | m (95% CI)^{[a]} | Tc (° C., 95% CI) |
|---|---|---|
| V₄₀ | −3.701 (−4.888-−2.714) | 51.6 (49.2-54.0) |
| V₄₀-Fr | −3.132 (−3.874-−2.391) | 36.4 (34.9-37.8) |
| (V/A)₈₀ | −4.445 (−6.403-−2.487) | 61.0 (58.5-63.5) |
| (V/A)₈₀-Fr | −4.238 (−5.467-−3.010) | 47.5 (43.6-51.4) |

^{[a]}° C./ln (μM/μM). 95% confidence intervals are calculated from the linear regression analysis using Graphpad prism.

TABLE 4
The thermodynamic parameters for the LLPS of V40, V40-
Fr, (V/A)80, and (V/A)80-Fr calculated from DSC curves.

| | | AS (mean ± SD, cal/ |
|---|---|---|
| Constructs | ΔH (mean ± SD, kcal/mol)a | (mol · K))a,b |
| V40 | 51.3 ± 5.2 | 701.8 ± 71.2 |
| V40-Fr | 29.8 ± 1.5 | 418.9 ± 20.8 |
| (V/A)80 | 66.2 ± 3.9 | 878.6 ± 53.3 |
| (V/A)80-Fr | 38.2 ± 0.3 | 521.3 ± 4.6 |

a(n = 2);
bcalculated from DS = DH/Tt.

TABLE 5
Hydrodynamic size and polydispersity index derived from the analysis of
autocorrelation functions using cumulants methods.

| | V40 | | V40-Fr | | (V/A)80 | | (V/A)80-Fr | |
|---|---|---|---|---|---|---|---|---|
| T | Zavg | | Zavg | | Zavg | | Zavg | |
| (° C.) | (nm)a | PdIa | (nm) | PdI | (nm) | PdI | (nm) | PdI |
| 15 | 10 ± 1 | 0.5 ± 0.0 | 57 ± 19 | 0.5 ± 0.0 | 11 ± 0.1 | 0.1 ± 0.0 | 61 ± 2 | 0.7 ± 0.0 |
| 20 | 11 ± 0.8 | 0.5 ± 0.0 | 55 ± 17 | 0.5 ± 0.0 | 14 ± 4 | 0.1 ± 0.0 | 54 ± 7 | 1 ± 0.0 |
| 25 | 10 ± 0.9 | 0.4 ± 0.0 | 51 ± 12 | 0.5 ± 0.0 | 169 ± 3 | 0.1 ± 0.0 | 51 ± 10 | 1 ± 0.0 |
| 30 | 12 ± 3 | 0.2 ± 0.1 | 730 ± 53 | 0.2 ± 0.0 | 16 ± 0.4 | 0.1 ± 0.0 | 59 ± 12 | 0.7 ± 0.1 |
| 35 | 14 ± 5 | 0.2 ± 0.1 | 1599 ± 56 | 0.1 ± 0.0 | 17 ± 2 | 0.1 ± 0.0 | 65 ± 16 | 0.7 ± 0.1 |
| 40 | 245 ± 102 | 0.3 ± 0.1 | 1613 ± 270 | 0.3 ± 0.2 | 18 ± 2 | 0.1 ± 0.0 | 1589 ± 1335 | 0.4 ± 0.1 |
| 45 | 1146 ± 339 | 0.2 ± 0.0 | 1704 ± 354 | 0.5 ± 0.1 | 75 ± 62 | 0.4 ± 0.1 | 3853 ± 1666 | 0.8 ± 0.2 |
| 50 | 2468 ± 772 | 0.5 ± 0.1 | 1782 ± 300 | 0.5 ± 0.4 | 519 ± 10 | 0.2 ± 0.0 | 3988 ± 2140 | 0.9 ± 0.1 |
| 55 | 2799 ± 840 | 0.5 ± 0.3 | 1837 ± 166 | 0.3 ± 0.1 | 3350 ± 1060 | 0.4 ± 0.1 | 5303 ± 2485 | 1 ± 0.1 |
| 60 | 3316 ± 50 | 0.4 ± 0.2 | 1829 ± 239 | 0.4 ± 0.4 | 4587 ± 484 | 0.7 ± 0.2 | 5048 ± 2359 | 1 ± 0.2 |
| 65 | 2987 ± 790 | 0.6 ± 0.5 | 1882 ± 288 | 0.2 ± 0.1 | 4134 ± 650 | 0.3 ± 0.1 | 4988 ± 2116 | 1 ± 0.0 |

amean ± SD (2 independent samples, each measured in triplicate).

What is claimed is:

1. A recombinant organism for forming a lipidated protein, comprising a host organism modified to include a first gene expressing an alpha subunit and a beta subunit of a transferase that will attach a lipid from a lipid donor to a protein and to include a second gene expressing the protein, wherein the protein includes a substrate of the transferase.

2. The recombinant organism of claim 1, wherein the transferase is selected from the group consisting of farnesyltransferase and geranylgeranyl transferase.

3. The recombinant organism of claim 2, wherein the substrate of the transferase comprises a first amino acid that is cysteine, a second amino acid that is hydrophobic, a third amino acid that is hydrophobic, and a fourth amino acid that has selectivity to farnesyltransferase or geranylgeranyl transferase.

4. The recombinant organism of claim 3, wherein the host organism is capable of endogenously producing the lipid donor.

5. The recombinant organism of claim 4, wherein the lipid donor comprises farnesyl pyrophosphate.

6. The recombinant organism of claim 5, wherein the substrate of the transferase is selected from the group consisting of SEQ ID NO: 2 and SEQ ID NO: 3.

7. The recombinant organism of claim 6, wherein the alpha subunit and the beta subunit are translationally coupled.

8. The recombinant organism of claim 7, wherein the alpha subunit and the beta subunit are translationally coupled by a stop codon of the beta subunit overlapped with a start codon of the alpha subunit.

9. The recombinant organism of claim 8, wherein the host organism comprises a bacterium.

10. The recombinant organism of claim 9, wherein the host organism comprises E. coli.

11. A method of producing a lipidated protein, comprising:
modifying a host organism to include a first gene expressing an alpha subunit and a beta subunit of a transferase that will attach a lipid from a lipid donor to a protein;
modifying the host organism to include a second gene expressing the protein, wherein the protein includes a substrate of the transferase;
providing the lipid donor; and
culturing the host organism to express the first gene and the second gene in the presence of the lipid donor to form the lipidated protein.

12. The method of claim 11, wherein the transferase is selected from the group consisting of farnesyltransferase and geranylgeranyl transferase.

13. The method of claim 12, wherein the substrate of the transferase comprises a first amino acid that is cysteine, a second amino acid that is hydrophobic, a third amino acid that is hydrophobic, and a fourth amino acid that has selectivity to farnesyltransferase or geranylgeranyl transferase.

14. The method of claim 13, wherein the host endogenously produces the lipid donor.

15. The method of claim 14, wherein lipid donor comprises farnesyl pyrophosphate.

16. The method of claim 15, wherein the substrate of the transferase is selected from the group consisting of SEQ ID NO: 2 and SEQ ID NO: 3.

17. The method of claim 16, wherein the alpha subunit and the beta subunit are translationally coupled.

18. The method of claim 17, wherein the alpha subunit and the beta subunit are translationally coupled by a stop codon of the beta subunit overlapped with a start codon of the alpha subunit.

19. The method of claim 18, wherein the host organism comprises a bacterium.

20. The method of claim 19, wherein the host organism comprises E. coli.


SEQUENCE LISTING (13 sequences)

SEQ ID NO: 1 - skipped

SEQ ID NO: 2 - protein, 4 aa, synthetic construct
CVLS

SEQ ID NO: 3 - protein, 4 aa, synthetic construct
CVLL

SEQ ID NO: 4 - protein, 5 aa, synthetic construct
GVGVP

SEQ ID NO: 5 - protein, 5 aa, synthetic construct
GZGVP

SEQ ID NO: 6 - skipped

SEQ ID NO: 7 - protein, 4 aa, synthetic construct
GCVL

SEQ ID NO: 8 - protein, 5 aa, synthetic construct
GCVLL

SEQ ID NO: 9 - protein, 205 aa, synthetic construct
GVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGVGVPGCVLL

SEQ ID NO: 10 - protein, 429 aa, synthetic construct
GVGVPGVGVPGAGVPGVGVPGVGVPGVGVPGVGVPGAGVPGVGVPGVGVPGGKGVGVPGVGVPGAGVPGVGVPGVGVPGVGVPGVGVPGAGVPGVGVPGVGVPGGKGVGVPGVGVPGAGVPGVGVPGVGVPGVGVPGVGVPGAGVPGVGVPGVGVPGGKGVGVPGVGVPGAGVPGVGVPGVGVPGVGVPGVGVPGAGVPGVGVPGVGVPGGKGVGVPGVGVPGAGVPGVGVPGVGVPGVGVPGVGVPGAGVPGVGVPGVGVPGGKGVGVPGVGVPGAGVPGVGVPGVGVPGVGVPGVGVPGAGVPGVGVPGVGVPGGKGVGVPGVGVPGAGVPGVGVPGVGVPGVGVPGVGVPGAGVPGVGVPGVGVPGGKGVGVPGVGVPGAGVPGVGVPGVGVPGVGVPGVGVPGAGVPGVGVPGVGVPGGKGCVLL

SEQ ID NO: 11 - protein, 376 aa, Rattus norvegicus
AATEGVGESAPGGEPGQPEQPPPPPPPPPAQQPQEEEMAAEAGEAAASPMDDGFLSLDSPTYVLYRDRAEWADIDPVPQNDGPSPVVQIIYSEKFRDVYDYFRAVLQRDERSERAFKLTRDAIELNAANYTVWHFRRVLLRSLQKDLQEEMNYIIAIIEEQPKNYQVWHHRRVLVEWLKDPSQELEFIADILNQDAKNYHAWQHRQWVIQEFRLWDNELQYVDQLLKEDVRNNSVWNQRHFVISNTTGYSDRAVLEREVQYTLEMIKLVPHNESAWNYLKGILQDRGLSRYPNLLNQLLDLQPSHSSPYLIAFLVDIYEDMLENQCDNKEDILNKALELCEILAKEKDTIRKEYWRYIGRSLQSKHSRESDIPASV

SEQ ID NO: 12 - protein, 440 aa, Rattus norvegicus
ASSSSFTYYCPPSSSPVWSEPLYSLRPEHARERLQDDSVETVTSIEQAKVEEKIQEVFSSYKFNHLVPRLVLQREKHFHYLKRGLRQLTDAYECLDASRPWLCYWILHSLELLDEPIPQIVATDVCQFLELCQSPDGGFGGGPGQYPHLAPTYAAVNALCIIGTEEAYNVINREKLLQYLYSLKQPDGSFLMHVGGEVDVRSAYCAASVASLTNIITPDLFEGTAEWIARCQNWEGGIGGVPGMEAHGGYTFCGLAALVILKKERSLNLKSLLQWVTSRQMRFEGGFQGRCNKLVDGCYSFWQAGLLPLLHRALHAQGDPALSMSHWMFHQQALQEYILMCCQCPAGGLLDKPGKSRDFYHTCYCLSGLSIAQHFGSGAMLHDVVMGVPENVLQPTHPVYNIGPDKVIQATTHFLQKPVPGFEECEDAVTSDPATDQEEF

SEQ ID NO: 13 - protein, 379 aa, Rattus norvegicus
AATEDDRLAGSGEGERLDFLRDRHVRFFQRCLQVLPERYSSLETSRLTIAFFALSGLDMLDSLDVVNKDDIIEWIYSLQVLPTEDRSNLDRCGFRGSSYLGIPFNPSKNPGTAHPYDSGHIAMTYTGLSCLIILGDDLSRVDKEACLAGLRALQLEDGSFCAVPEGSENDMRFVYCASCICYMLNNWSGMDMKKAISYIRRSMSYDNGLAQGAGLESHGGSTFCGIASLCLMGKLEEVFSEKELNRIKRWCIMRQQNGYHGRPNKPVDTCYSFWVGATLKLLKIFQYTNFEKNRNYILSTQDRLVGGFAKWPDSHPDALHAYFGICGLSLMEESGICKVHPALNVSTRTSERLRDLHQSWKTKDSKQCSDNVHISSQEE